(19) United States
(12) Patent Application Publication
(10) Pub. No.: US 2025/0034629 A1
(43) Pub. Date: Jan. 30, 2025

(54) Method for Dynamic Range Expansion for Multiplex Assays

(71) Applicant: APPLIED BIOCODE INC., Santa Fe Springs, CA (US)

(72) Inventors: Winston Z. Ho, Hacienda Heights, CA (US); Shu Huang, Acadia, CA (US); Jerry Kowalski, Murrieta, CA (US); Gao Chen, Hawthorne, CA (US)

(21) Appl. No.: 18/225,479

(22) Filed: Jul. 24, 2023

(51) Int. Cl. C12Q 1/6837; B01L 3/00; G01N 33/543

(52) U.S. Cl. CPC C12Q 1/6837; B01L 3/502761; G01N 33/54313; G01N 33/54386; B01L 2200/0652; B01L 2200/16; B01L 2300/0654; C12Q 2600/16

(57) ABSTRACT

Techniques for conducting an assay for multiple analytes in a sample wherein the presence/absence of each analyte in said sample is indicated by a light signal are provided. An example method includes determining the presence of an analyte by: providing a light to the sample, wherein a light signal is emitted from the sample associated with the presence of individual analytes, capturing a plurality of images by one or more photodetectors, each including the plurality of analytes, at a plurality of durations, and for respective analytes of the plurality of analytes, determining a range of integration durations associated with a light signal associated with the presence of the analyte, identifying an image of the plurality of images, captured at a duration within the range of integration durations, and analyzing the image to determine the presence of the analyte.

### FIELD OF THE INVENTION

The invention relates generally to assay methods and systems for use thereof to carry out bioassays such as specific binding assays, and more particularly multiplexed bioassays for protein and nucleic acid analysis. Immunoassay and molecular assays often require a large dynamic range for quantitative or semi-quantitative assays. The invention generally relates to adjusting and expanding the dynamic ranges for all analytes in the multiplex assay panel to match the intrinsic electronic dynamic range of the detector.

### BACKGROUND OF THE INVENTION

### Description of Related Art

### Multiplex Assays

A growing demand for specific binding assays including immunoassays and molecular assays has put pressure on the diagnostic industry to reduce costs and optimize sample usage by designing multiplex assays to test for several analytes simultaneously. Instead of performing one test per sample by the conventional method, many tests can be conducted which generate many data points per sample. Factor in the time saved by running the equivalent of 20, 100, or more different assays in a single assay workflow, and the benefits of multiplexing are clear. For example, the usage of 20 microplates, one plate per test, to run 20 tests, can be reduced to 1 microplate producing significant savings in manufacturing costs. An increase in testing volume and a pressure of rapid turnaround results further enhance the value of multiplex assays. The ability to measure multiple targets in a single assay allows researchers and laboratory personnel to detect hundreds of analytes and generate thousands of test data points in a single run. While being judicious with precious sample volumes, it is known that it is difficult to get sufficient blood samples from many pediatric patients. From screening biomarkers for further evaluation to validating results for drug development, multiplex assays provide many important benefits, helping to save time, save labor, save material resources, conserve limited sample volume, reduce test cost, and optimize productivity.

The presence or absence of analyte in a sample is determined by contacting the sample with agents which will specifically bind to the desired analyte but not with other analytes which have been practiced for decades and are well known in the art. The presence or absence of a binding reaction can be determined by use of light signals to detect the presence or absence of the specific binding pair. There are multiple multiplex technologies, such as color coded microbead, digitally coded magnetic bead, microarray, and multiple color fluorophores. In the multiplex assay, the coded micro particles are used for analyte identification. The number of analytes measured is determined by the number of different bead colors, digital barcodes, or micro spots in the microarray. Multiplex assays within a given application area or class of technology can be further stratified based on how many analytes can be measured per assay, where “multiplex” refers to those with the highest number of analyte measurements per assay. The “low-plex” (>2 and <5) or “mid-plex” refers to procedures that process fewer (>5 and <50), though there are no formal guidelines for calling a procedure multi-, mid-, or low-plex based on number of analytes measured. In the biological sciences, a multiplex assay can use magnetic beads to simultaneously measure multiple analytes in a single experiment. A multiplex immunoassay is one form of a specific binding assay and is a derivative of an ELISA using beads for binding the capture antibody. In the past few years, multiplex assays have started to migrate from research applications into clinical settings.

### Sensor Dynamic Range

Many factors can affect the dynamic range of bioassays, such as the power of light source, sample volume, matrix volume or matrix surface area, particle or bead number, electronic gain factors, optical elements, and quality and nature of optical detectors. Among them, optical detectors play a very significant role in term of dynamic range. Most of the quantitative biological tests are performed using an optical sensor or detector, such as CCD, CMOS, photo multiplied tube or the like. The sensor chip is the key element of bioassay detection, but the intrinsic dynamic range of the sensor is limited by its electronic capacity.

For example, Charge Coupled Device (CCD) is a highly sensitive photon detector. The sensor chip is divided up into many light-sensitive small areas (known as pixels) which can be used to build up an image of the scene of interest. A photon of light which falls within the area defined by one of the pixels will be converted into one (or more) electrons and the number of electrons collected will be directly proportional to the intensity of the scene at each pixel. When the sensor is clocked out (i.e., the integration duration is run), the number of electrons in each pixel is measured and the number of photons can be reconstructed. For example, the dynamic range of a CCD is typically defined as the full-well capacity divided by the camera noise and relates to the ability of a camera to record simultaneously very low light signals alongside bright signals. The ratio is often expressed in decibels which is calculated as DR (full well capacity/read noise) or in the equivalent number of A/D units required to digitize the signal. Because all optical sensors can reach photon saturation at full-well capacity, the sensor ability of the sensor to expand the upper end of the dynamic range is limited. At the same time electronic noise limits the lower end of the dynamic range. Because the electronic dynamic range of a sensor is fixed. The dynamic range (DR) of every analyte in the sample need to fall into or be able to match the DR of the sensor in order for its presence and/or quantity to be accurately assessed.

### Dynamic Ranges of Bioassays and Multiplex Assays

Bioassays, both immunoassay and molecular specific binding assays often require a large dynamic range for quantifying analyte with a wide range of analytical concentrations. The need becomes even more challenging for multiplex assays, in which the multiple analytes may have very different range of concentrations in a sample. For single analyte target, sample dilutions are commonly method to adjust the analytical concentration for detection purpose, but it becomes impossible to adjust this accurately for multiple analytes having significantly different concentration and/or signal ranges. Every individual target in the multiplex assays needs to operate within its own proper dynamic range for quantitative assays, but the range of concentration can vary significantly.

Bioassays often require a large dynamic range for quantifying analyte with a wide range of concentrations. For bioassays, the dynamic range of an assay is described as the lowest to the highest concentration of an analyte that can be reliably detected by the assay. This is referred to as the lower and upper limits of detection (LLOD and ULOD, respectively). With a commonly available bioassay systems, the useful dynamic range is limited to approximately 2 to 4 decades (2 log-4 log).

For detection of a single target, dilutions are commonly used to adjust the analyte concentration in the sample to be detected by the detector. The concentration of the analyte may be very different from sample to sample under different clinical conditions. It is important to have a large dynamic range to cover concentration ranges of clinical significance. Although sample dilutions have become common practice, it becomes very difficult to carry out appropriate dilutions for multiple analytes in one sample. The concentration range of interest for an individual analyte can vary widely, for example, in the immunoassays: Analyte 1: from 0.1 pg/ml to 1,000 pg/ml, Analyte 2: from 1.0 pg/ml to 10,000 pg/ml, 3: from 10 pg/ml to 100,000 pg/ml etc. In the nucleic acid, DNA, RNA molecular assays: the analyte concentrations can be ranged from a single copy to 1,000 s of copies. The quantitation can be even more difficult for post PCR reactions, it can range from 0.001 fmole/μl to 1,000 fmole/μl with a wide dynamic range. If the sample for one highly concentrated analyte is diluted it may become impossible to detect other analytes with low concentrations. Considering the number of analytes in the multiplex assays, it becomes a daunting task to have proper dilutions, wherein all analytes of all samples fall into the dynamic range of the single sensor.

### SUMMARY OF THE INVENTION

A method and system are provided with adjustable and expandable dynamic ranges for two or more of the analytes in multiplex bioassays using a plurality of optical integration durations. Depending on the analyte, a short exposure time is used to avoid sensor saturation in order to extend the upper limit of the concentration. To extend the lower limit of the concentration, a long exposure time is used to generate more photons and overcome the read noise. In this manner, the dynamic range for detection of each analyte in multiplex assays can be optimized and customized.

The present invention is directed to enhancing the dynamic range of bioassays for quantitative measurements. More importantly, it solves the problems of limited dynamic ranges for multiplex assays, in which each target analyte has a wide range of concentration range. Because all optical sensors are limited by full-well electron capacity for the upper limit of the dynamic range and electronic noise for the lower end of the dynamic range the extent of the dynamic range of the sensor is a fixed number. However, the amount of photons detected by the sensor depends on the time duration of the photon flux generated by the analyte. In the case of fluorescence, the total measurable fluorescence intensity is the multiplied product of “analyte concentration” and “time duration of the light intensity”.

The present invention is directed to obtain a plurality of signals from a series of integration durations for every measurement. A short integration duration is used to generate a smaller number of photons to avoid the sensor saturation for high concentration; and a long integration duration is used to generate a greater number of photons to overcome the detector noise for low concentration. A series of integration durations is thus utilized for each sample measurement.

In an embodiment, a method of conducting an assay for multiple analytes in a sample, wherein the presence or absence of each analyte in said sample is indicated by a light signal, is provided. The method may include the steps of: determining the presence of an analyte by: providing a light source to the sample wherein a light signal is emitted associated with the presence of individual analytes; capturing, by one or more photodetectors, a plurality of images, each including the plurality of analytes, at a plurality of durations; for a first analyte of the plurality of analytes, determining, by one or more processors, a first range of integration durations associated with a light signal associated with the presence of the first analyte; identifying, by the one or more processors, a first image, of the plurality of images, captured at a duration within the first range of integration durations associated with the light signal associated with the presence of the first analyte; and analyzing, by the one or more processors, the first image to determine the presence of the first analyte; and for a second analyte of the plurality of analytes, determining, by one or more processors, a second range of integration durations associated with the presence of the second analyte; identifying, by the one or more processors, a second image, of the plurality of images, captured at a duration within the second range of integration durations associated with the light signal associated with the presence of the second analyte; and analyzing, by the one or more processors, the second image to determine the presence of the second analyte.

In another embodiment, a method of conducting a specific binding assay for multiple analytes in a sample wherein the presence or absence of each analyte in said sample is indicated by a light signal, is provided. The method may include providing a specific binding pair member for each analyte, contacting the sample with the specific binding pair members under conditions in which analytes will stably bind with their specific binding pair member but will not bind to other specific binding pair members and in which stably bound specific binding pairs are capable of being detected by emitting a light signal determining the presence of stably bound specific binding pair members by: providing a light source to the plurality of specific binding pair members and analytes over a period of time wherein stably bound specific binding pairs will emit a light signal; capturing, by one or more photodetectors, a plurality of images, each including the plurality of analytes, at a plurality of durations; for a first analyte of the plurality of analytes, determining, by one or more processors, a first range of integration durations associated with a light signal associated with the presence of the first analyte; identifying, by the one or more processors, a first image, of the plurality of images, captured at a time within the first range of integration durations associated with the light signal associated with the presence of the first analyte; and analyzing, by the one or more processors, the first image to determine the presence of the first analyte; and for a second analyte of the plurality of analytes, determining, by one or more processors, a second range of integration durations associated with the presence of the second analyte; identifying, by the one or more processors, a second image, of the plurality of images, captured at a time within the second range of integration durations associated with the light signal associated with the presence of the second analyte; and analyzing, by the one or more processors, the second image to determine the presence of the second analyte.

In yet another embodiment, a method for detecting a signal associated with the quantitative measurement of an analyte in a multiplex bioassay, wherein measuring the concentration of an analyte in a sample is indicated by a light signal strength, is provided. The method may include identifying each analyte, of a plurality of analytes on solid substrates in a multiplex bioassay, by optical imaging; capturing a plurality of signals, N, at each of a series of integration durations, IT-N, for each analyte in said multiplex assay, wherein each image contains light signals for each analyte in the multiplex assay; for each respective analyte, selecting a respective light signal, of the plurality of light signals, captured at a respective integration duration, of the series of integration durations, that is within a linear range associated with the respective analyte; normalizing the other light signals, of the plurality of light signals by compensating for their integration durations based on a ratio of their integration durations to the respective integration duration that is within the linear range associated with the respective analyte; and combining the normalized light signals in order to expand the linear range associated with the respective analyte of the plurality of analytes.

In still another embodiment, a system with an analyte-dependent dynamic range for measuring a concentration of each analyte, of a plurality of analytes in a multiplex bioassay, may be provided, comprising: a light source configured to provide a series of pulses having different time durations to excite multiple analytes on solid substrates in said sample; an imaging detector configured to capture a plurality of light image signals (N), having a respective plurality of optical integration durations (ID-N) corresponding to the pulses of said light source, wherein each of said light image contains light signals for each analyte in said multiplex bioassay; one or more processors; and one or more memories storing instructions that, when executed by the one or more processors, cause the one or more processors to, for each analyte of the plurality of analytes: for each respective analyte, select a respective light signal, of the plurality of light signals, captured at a respective integration duration, of the series of integration durations, that is within a linear range associated with the respective analyte; normalize the other light signals, of the plurality of light signals by compensating for their integration durations based on a ratio of their integration durations to the respective integration duration that is within the linear range associated with the respective analyte; and combine the normalized light signals in order to expand the linear range associated with the respective analyte of the plurality of analytes.

### BRIEF DESCRIPTION OF THE DRAWINGS

For an understanding of the scope and nature of the invention, as well as the preferred mode of use, reference should be made to the following detailed description read in conjunction with the accompanying drawings. In the following drawings, which are examples, like reference numerals designate like or similar parts throughout the drawings.

FIG. 1. illustrates a principle of the present invention with a series of illumination light pulses, and a series of fluorescence light pulses which are detected by a plurality of integration durations for detecting a train of photo flux.

FIG. 2. shows a typical example of fluorescence signal versus analyte concentration for of one integration duration with a limited dynamic range with a log of 2 to 3 order of magnitude,

FIG. 3. illustrates an example of fluorescence signal versus analyte concentration of one integration duration with the sensor saturation, it limits the high-end part of the dynamic range.

FIG. 4. illustrates an example of two integration durations: the first integration duration (the top curve) with the sensor saturation, it limits the high-end part of the dynamic range and the second integration duration with short time duration (the bottom curve).

FIG. 5. illustrates a real experimental data of fluorescence signal versus analyte concentration with two integration durations: the top curve shows the first integration duration with the sensor saturation, it limits the high-end part of the dynamic range, and the bottom curve shows the data with the second shorter integration duration.

FIG. 6. illustrates fluorescence signal versus analyte concentration with two integration durations: the top curve shows the first integration duration with the sensor saturation, it limits the high-end part of the dynamic range; while the bottom curve shows the data with the second, but shorter integration duration. The data of the second integration duration data is normalized to the first integration duration; and by combine two set of signals operating in linearity to expand the dynamic range.

FIG. 7. illustrates an example of fluorescence signal versus analyte concentration of two integration durations: first integration duration with the sensor limits the low-end part of the dynamic range, and a third integration duration, with long time duration, to improve the signal at limit of the detection.

FIG. 8 illustrates a block diagram of an example system for implementing the techniques provided herein.

FIG. 9 illustrates a flow diagram of an example method of conducting an assay for multiple analytes in a sample wherein the presence or absence of each analyte in said sample is indicated by a light signal, as may be implemented via the system shown at FIG. 8, in accordance with some embodiments provided herein.

FIG. 10 illustrates a flow diagram of an example method of conducting a specific binding assay for multiple analytes in a sample wherein the presence or absence of each analyte in said sample is indicated by a light signal, as may be implemented via the system shown at FIG. 8, in accordance with some embodiments provided herein.

FIG. 11 illustrates a flow diagram of an example method of detecting a signal associated with the quantitative measurement of an analyte in a multiplex bioassay, wherein measuring the concentration of an analyte in a sample is indicated by a light signal strength, as may be implemented via the system shown at FIG. 8, in accordance with some embodiments provided herein.

### DETAILED DESCRIPTION OF EMBODIMENTS OF THE INVENTION

In order to conduct quantitative and semi-quantitative biological assays it is important to have a large dynamic range to cover all analytes and samples for all patients under all health conditions. Recently, multiplex assays, which offer multiple tests have become very popular and have offer tremendous advantages over the conventional one test per sample. However, for multiplex assays, it also creates challenges for quantitative assays because the dynamic range needed to cover all the analytes. Considering the number of analytes in the multiplex assays, it becomes a daunting task. Conventional wisdom which is to dilute a sample to a certain concentration, and let all analytes fall into the single set of dynamic range of the single sensor becomes impractical to carry out because of the wide range of analyte concentrations and resulting signals ranging over many logs. Accordingly, a method and system which can adjust and expand the dynamic ranges depend on each of the analyte is needed.

The present invention is directed to a method for adjustable and expandable dynamic ranges for multiplex bioassays is comprising a) detecting a plurality of signals, N, from a series of optical integration durations, IT-N, for each analyte in said multiplex assays; b) normalizing each of multiple signals to a corresponding integration duration or normal integration duration; and c) selecting and combining some of the plurality of the normalized signals operating in linearity to adjust and expand the dynamic range

The present invention is directed to a method for adjustable and expandable dynamic ranges for multiplex bioassays wherein the plurality of signals, N=2-5 signals.

The present invention is directed to a method for adjustable and expandable dynamic ranges for multiplex bioassays of wherein the series of optical integration durations range from 0.1 millisecond to 10 second but can also range from 1 millisecond to 1 second, to from 10 milliseconds to 0.1 seconds. The multiple signals are obtained from optical sensors, comprising CCD, CMOS camera, photo diode, and photomultiplier tubes. The bioassay comprises specific binding assays including but not limited to immunoassays or molecular assays. The plurality of signals can be of any light spectrum including and outside of the human visual spectrum and according to preferred aspects of the invention include fluorescent or chemiluminescent signals.

The present invention is directed to a system for adjustable and expandable dynamic ranges for multiplex bioassays comprising a) a light source generating a plurality of excitation lights with different time duration; b) a sensor detecting a plurality of signals with a series of integration durations; c) a processor normalizing the plurality of signals from the sensor according to a corresponding integration duration; and selecting and combining a plurality of the normalized signals operating in linearity to adjust and expand the dynamic range for each analyte in the multiplex assays.

The present invention is directed to a system for adjustable and expandable dynamic ranges for multiplex bioassays wherein the plurality of signals, N=2-5 signals.

The present invention is directed to a system for adjustable and expandable dynamic ranges for multiplex bioassays of wherein the series of optical integration durations range from 0.1 millisecond to 10 second as well as other narrower and broader ranges. The multiple signals are obtained from optical sensors, comprising CCD, CMOS camera, photo diode, and photomultiplier tubes. The bioassay comprising immunoassays or molecular assays. The plurality of signals is fluorescence or chemiluminescence signal.

The present invention is directed to a system for adjustable and expandable dynamic ranges for multiplex bioassays, the multiple optical integration durations are in synchronizing with the excitation of the light source.

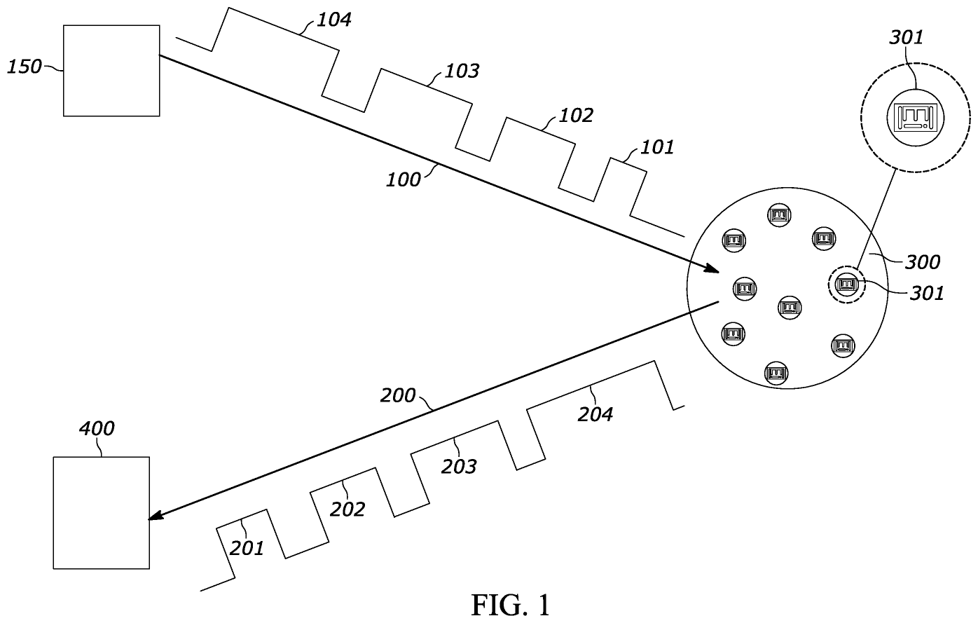

FIG. 1. illustrates the principle of the present invention with a series of illumination light pulses, and a series of fluorescence light pulses which are detected by a plurality of integration duration for detecting a train of photo flux. FIG. 1 show excitation light 100 from a light source 150, with a series of excitation light pulses N, such as pulse 101, 102, 103, 104, etc., ranging from 0.01 millisecond to 10 second. For example, the excitation light pulse 101, 102, 103, 104 represent a pulse duration of 1 millisecond (ms), 10 ms, 100 ms, 300 ms, 500 ms (unshown), respectively. The sample solution 300 comprising of solid phase micro particles or micrometer size magnetic beads 301. After the biological reactions, the positive sample is labeled with fluorophores according to methods well known in the art. Since the typical fluorescence lifetime is very short, in the range of nanoseconds or microseconds, thus the fluorescence light 200 is in sync with the excitation light. A series of fluorescence light pulses, 201, 202, 203, 204, etc. are generated from the fluorophore, and consequently detected by a detector 400, which integrates the fluorescence signals with a gated integrated time, IT-N, ranging from 0.01 millisecond to 10 second. A series of integration durations, N, for example: IT-1=1 ms, IT-2=10 ms, IT-3=100 ms, IT-4=300 ms, IT-5=500 ms, etc. The signal integration duration typically is either shorter or the same duration as the fluorescence light pulses. The intensity of the fluorescence signal is proportional to the duration of the integration duration. The longer the integration duration, the greater the signal. Therefore, by adjusting the integration duration, it is possible to control the photo flux, to increase the sensitivity at the lower end, to avoid the sensor saturation at the high-end, and thus, to expand the dynamic ranges. The number of integration duration, N, is preferably in the range of 2-5, each with different time duration from low to high but can be greater if appropriate as determined by the practitioner for the given situation.

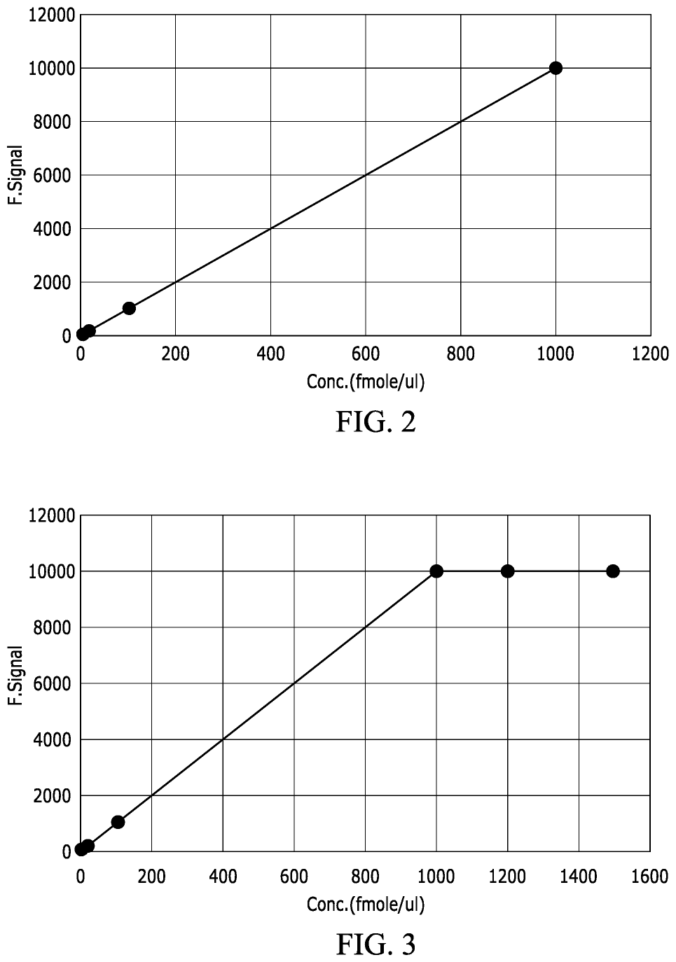

The concentration range of interest for individual analyte can vary widely. For example, for an immunoassay, it can be ranged from 0.01 pg/ml to 100,000 pg/ml; and for nucleic acid assays, from 0.001 to 1,000 fmole/μl. FIG. 2. shows an example of fluorescence signal versus analyte concentration for of one integration duration, for example 100 ms, a limited dynamic range with a log of 2 to 3 order of magnitude, range from 1 to 1,000 fmole/μl, but when the concentration is less than 50 fmole/μl, the signal is buried in the detector noise. The fluorescence intensity unit is commonly represented by Medium Fluorescence Intensity (MFI) ranged from 10 to 100,000 MFI. At upper end of the detection, when the intensity is more than 10,000 MFI, the sensor is saturated. While at lower end of the detection, when the intensity is less than 50 MFI, it become non-detectable. FIG. 3. illustrates an example of one integration duration with sensor saturation, it limits the high-end part of the dynamic range. When the concentration is more than 1,000 fmole/μl, the signal is more than 10,000 MFI, the sensor is saturated. This is a common problem; it reaches the sensor detection capacity.

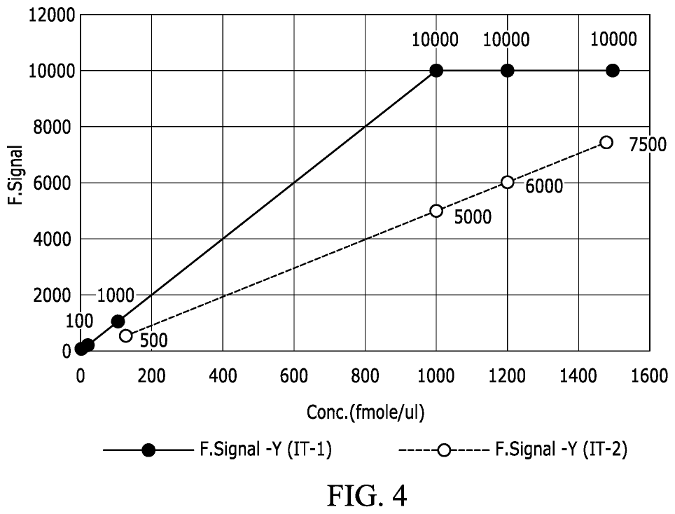

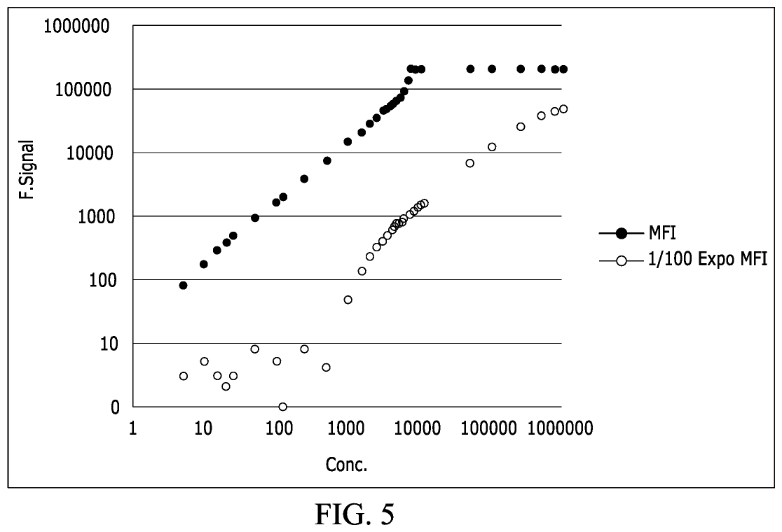

FIG. 4. illustrates an example of two integration durations: the top curve shows the first integration duration, e.g. IT-1=100 ms, with the sensor saturation. It limits the high-end part of the dynamic range beyond the concentration of 1,000 fmole/μl with 10,000 MFI. The bottom curve shows the second shorter integration duration, e.g. IT-2=50 ms, with signal of 5000, 6000, and 7500 MFI at high-end, which are below the signal saturation of 10,000 MFI with the concentrations of 1,200 and 1,500 fmole/μl. FIG. 5. illustrates a real experimental data of fluorescence signal versus analyte concentration with two integration durations: the top curve shows the sensor saturation, with the first integration duration of 100 ms, which limits the high-end part of the dynamic range. The bottom curve, with the second shorter integration duration of 1 ms, shows the fluorescence signal is almost 100 time less than the long integration duration. The upper end of the dynamic range, 10,000 to 100,000 fmole/μl, extends near 100 folds beyond the 1,000 fmole/μl, which is the upper limit of the long integration duration.

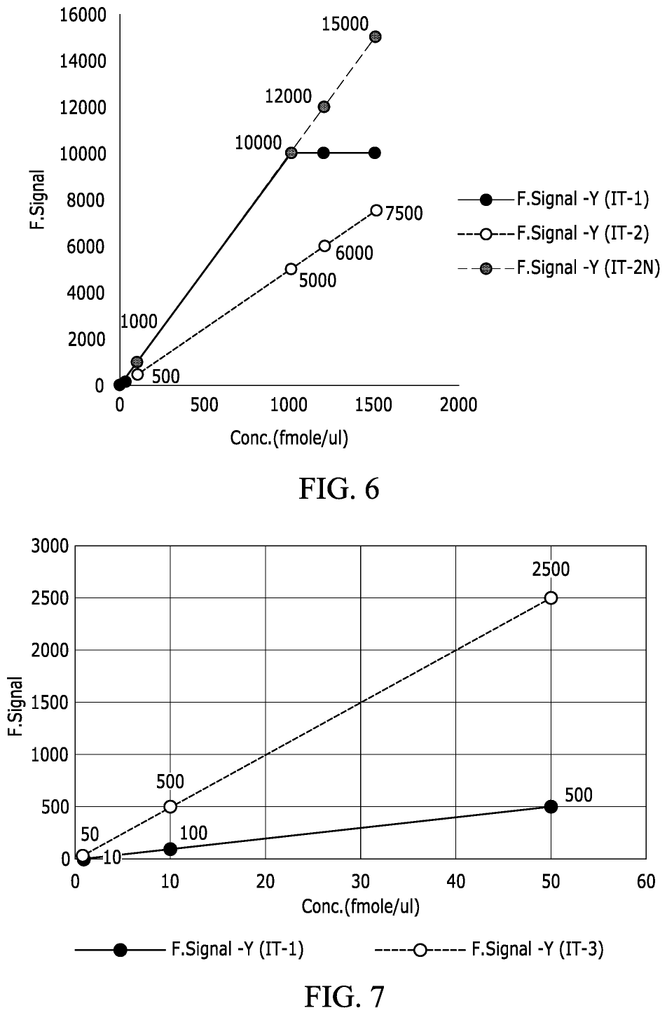

For quantitative assays, a calibration curve: signal vs. concentration trendline, operating in a linearity is preferred to provide better accuracy for data interpretation. FIG. 6. illustrates fluorescence signal versus analyte concentration with two integration durations: the top curve shows the first integration duration, IT-1=100 ms, with the sensor saturation, it limits the high-end part of the dynamic range, and the bottom curve shows the data with the second shorter integration duration, IT-2=50 ms. The second integration duration data is normalized to the first or corresponding integration duration (x 100 ms/50 ms), the normalized signal become 10,000, 12,000, and 15,000 MFI from the original 5,000, 6,000, and 7,500 MFI. By selecting, in this case IT-1 and IT-2, and combining two set of signals operating in linearity, the upper end of the dynamic range is extended from the original 1,000 fmole/μl to 1,200 and 1,500 fmole/μl.

While the read noise or electronic noise limits the lower end of the dynamic range. High sensitivity is often needed for analytes having very low concentrations. This is referred to as the lower limits of detection (LLOD). The LLOD is often limited by the sensor read noise. To be considered as good signal, the signal/noise ratio should be >3. However, the optical signal depends on the number of photons being generated and being detected. If we increase the duration of the excitation time and the corresponding integration duration, the sensor can detect more photons, thus improving sensitivity. FIG. 7. illustrates an example of fluorescence signal versus analyte concentration of third integration durations: first integration duration of TI-1=100 ms and third integration duration of TI-3=500 ms. The signals for TI-3 at the low end of the limit has 50, 500, and 2,500 MFI, in comparison to the signals for IT-1 with 10, 100, and 500 MFI. When the read noise is between 5-10 MFI, a signal value of <30 is often considered not detectable. The number of photons generated by the analyte must be higher than the limit of the electronic noise of the sensor. By increasing the integration duration from 100 ms to 500 ms, the lower end of the dynamic range is extended from 10 fmole/μl to 1 fmole/μl.

By combining a plurality of integration durations, N, normalizing the signal to a corresponding integration duration (or normally use standard integration duration), selecting some sets, N=2-5, of the normalized signals which operating in linearity, in terms of signal versus concentration, thus the dynamic range of each analyte in the multiplex assays can be adjusted and expanded.

### Example System

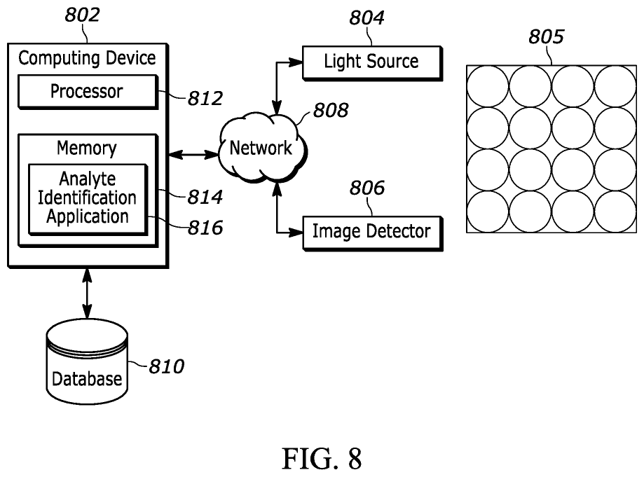

FIG. 8 depicts an exemplary system 800 for implementing the techniques provided herein, in accordance with some embodiments provided herein. The high-level architecture illustrated in FIG. 8 may include both hardware and software applications, as well as various data communication channels for communicating data between the various hardware and software components, as is described below.

The system 800 may include a computing device 802, as well as a light source 804 and an image detector 806 (e.g., which may include one or more of CCD, CMOS cameras, photo diodes, photomultiplier tubes, etc.). The light source 804 may be configured to provide light to analytes on solid substrates in a sample 805, and the image detector 806 may be configured to capture images of the analytes on the solid substrates in the sample 805.

For instance, a specific binding pair member may be provided for each analyte in the sample 805. For instance, the specific binding assay may be an antigen-antibody assay. As another example, the specific binding assay may be a nucleic acid hybridization assay. The nucleic acid hybridization assay may be a DNA hybridization assay, or an RNA hybridization assay, in various examples. The sample 805 may be contacted with specific binding pair members under conditions in which analytes will stably bind with their specific binding pair member but will not bind to other specific binding pair members and in which stably bound specific binding pairs are capable of being detected by emitting a light signal. For instance, the binding pair members may be bound to a solid substrate. The solid substrate may comprise a plurality of digitally coded beads, barcoded magnetic beads, color coded microbeads, or microarray settings on a planar surface. For instance, the beads may be labeled, e.g., by a bar code. In some examples, the beads may be magnetic. Additionally, in some examples, two or more specific binding pair members may be bound to different physical locations on the solid substrate.

For instance, in the example of barcoded solid substrates, each barcoded solid substrate may be immobilized with a specific biomolecular probe which can react with a specific analyte, but not other analytes, in the multiplex assay in the sample. The resulting analyte may be labelled with fluorophore for fluorescence detection.

The computing device 802, light source 804 and image detector 806 may be configured to communicate with one another via a wired or wireless network 810. To facilitate such communications, the computing device 802, light source 804, and image detector 806 may each respectively comprise a wireless transceiver to receive and transmit wireless communications.

In some embodiments, the computing device 802 may comprise one or more servers, which may comprise multiple, redundant, or replicated servers as part of a server farm. In still further aspects, such server(s) may be implemented as cloud-based servers, such as a cloud-based computing platform. For example, such server(s) may be any one or more cloud-based platform(s) such as MICROSOFT AZURE, AMAZON AWS, or the like. Such server(s) may include one or more processor(s) 812 (e.g., CPUs) as well as one or more computer memories 814.

Memories 814 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 122 may store an operating system (OS) (e.g., Microsoft Windows, Linux, UNIX, etc.) capable of facilitating the functionalities, apps, methods, or other software as discussed herein. Memorie(s) 814 may also store an analyte identification application 816.

Additionally, or alternatively, the memorie(s) 816 may store data from various sources. This data may also be stored in a database 810, which may be accessible or otherwise communicatively coupled to the computing system 802.

Executing the analyte identification application 816 may include controlling the light source 804 to provide light to the sample 805. In various examples, the light source 804 may provide visible light or ultraviolet light to the sample 805. Furthermore, in various examples, the light source 804 may continuously provide light to the sample 805 over a period of time, or may provide pulses of light of various durations to the sample 805. Light signals (e.g., fluorescence signals, chemiluminescence signals, etc.) may be emitted by the sample 805 when particular analytes are present in the sample 805. Executing the analyte identification application 816 may further include controlling the image detector 806 to capture images of the sample 805 at various integration durations. For instance, the image detector 806 may be controlled to capture images at various times over a time period during which the light source 804 continuously provides light to the sample 805, or may be controlled to capture images associated with various pulses of light provided by the light source 804 to the sample 805. For instance, the image detector 806 may capture images at integration durations ranging from 0.01 milliseconds to 10 seconds.

Executing the analyte identification application 816 may further include determining respective ranges of integration durations associated with light signals that indicate the presence of respective analytes in the sample 805. For instance, the analyte identification application 816 may calculate these ranges for each analyte based on a signal-concentration response curve for that analyte. These signal-concentration responses curves, or the ranges themselves, may be stored in the database 810 in some examples and accessed by the analyte identification application 816. For each respective analyte, the analyte identification application 816 may identify and/or select a respective one of the images captured by the image detector 806 that was captured at an integration duration within the range of integration durations associated with that analyte. The analyte identification application 816 may analyze the identified and/or selected image for a particular analyte in order to determine the presence or absence of that analyte (e.g., based on whether the image includes a light signal emitted by the analyte). This process may be repeated for two or more of the analytes, or for all of the analytes, that may be present in the sample 805.

Additionally, in some examples, analyte identification application 816 may further normalize the other light signals by compensating for their integration durations based on a ratio of their integration durations to the respective integration duration that is within the linear range associated with the respective analyte. The analyte identification application 816 may combine these normalized light signals in order to expand the linear range associated with the respective analyte of the plurality of analytes.

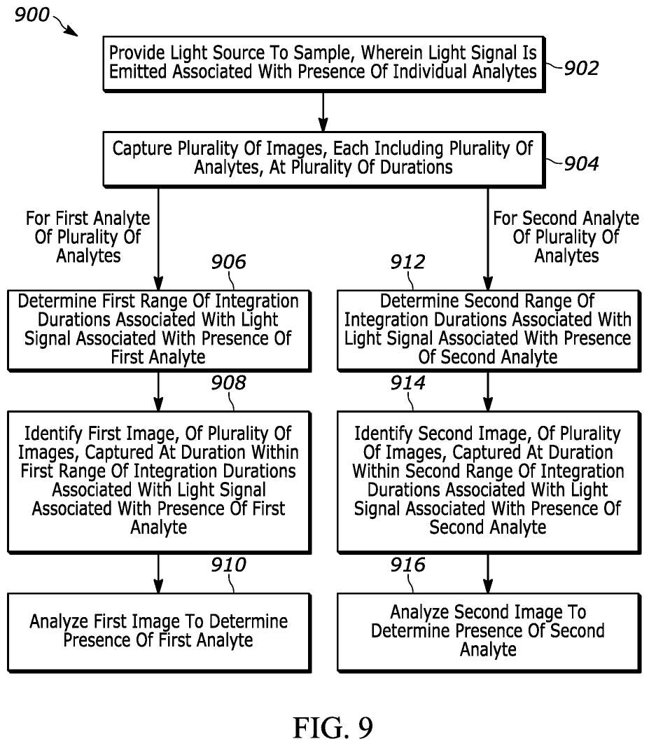

In addition, memories 814 may also store additional machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memory 814 may include instructions for carrying out any of the steps of any of the methods 900, 1000, or 1100 (which are described in greater detail below with respect to FIGS. 9, 10, and 11, respectively), via an algorithm executing on the processors 812. It should be appreciated that one or more other applications may be envisioned and that are executed by the processor(s) 814. It should be appreciated that given the state of advancements of mobile computing devices, all of the processes functions and steps described herein may be present together on a mobile computing device.

### Example Methods

FIG. 9 illustrates a flow diagram of an example method 900 of conducting an assay (e.g., a specific binding assay) for multiple analytes in a sample wherein the presence or absence of each analyte in said sample is indicated by a light signal, in accordance with some embodiments provided herein. One or more steps of the method 900 may be implemented as a set of instructions stored on a computer-readable memory (e.g., memory 814) and executable on one or more processors (e.g., processor 812). At block 902, a light source (e.g., the light source 804) may provide light to a sample (e.g., the sample 805) including a plurality of analytes. As discussed above with respect to the light source 804, the light source may provide visible light or ultraviolet light in various examples. Light signals may be emitted by the sample when particular analytes are present in the sample.

At block 904, a plurality of images may be captured by a light detector (e.g., the light detector 806). Each image may include the plurality of analytes in the sample, and each image may be captured at a different integration duration. That is, over a period of time during which the light source continuously provides light to the sample, the image detector may capture a plurality of images at different times during that period of time. Alternatively, the light source may provide pulses of light to the sample, with each pulse having a different duration, and the image detector may capture respective images of the sample associated with each pulse of light.

For a first analyte of the plurality of analytes, the method 900 may proceed from block 904 to blocks 906-910, and for a second (different) analyte of the plurality of analytes, the method 900 may proceed from block 904 to blocks 912-916. In some examples, the method 900 may proceed in a similar manner with a third analyte, fourth analyte, etc., of the plurality of analytes.

At block 906, for the first analyte of the plurality of analytes of the sample, the method 900 may include determining a first range of integration durations of the light signal associated with the presence of the first analyte. For instance, the first range may be calculated based on a signal-concentration response curve for the first analyte. At block 908, a first image that is captured at an integration duration within the first range of integration durations may be identified, and at block 910, the first image may be analyzed to determine the presence (or absence) of the first analyte.

At block 912, for the second analyte of the plurality of analytes of the sample, the method 900 may include determining a second range of integration durations of the light signal associated with the presence of the second analyte. For instance, the second range may be calculated based on a signal-concentration response curve for the second analyte. At block 914, a second image that is captured at an integration duration within the second range of integration durations may be identified, and at block 916, the second image may be analyzed to determine the presence (or absence) of the first analyte.

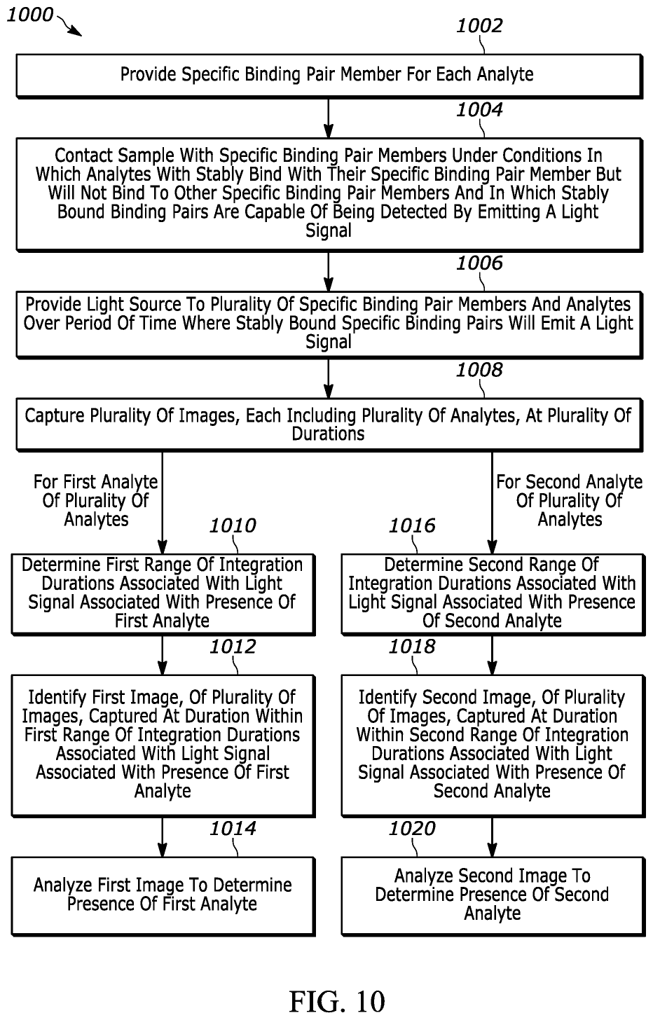

FIG. 10 illustrates a flow diagram of an example method 1000 of conducting a specific binding assay for multiple analytes in a sample (e.g., the sample 805) wherein the presence or absence of each analyte in said sample is indicated by a light signal, in accordance with some embodiments provided herein. One or more steps of the method 1000 may be implemented as a set of instructions stored on a computer-readable memory (e.g., memory 814) and executable on one or more processors (e.g., processor 812).

At block 1002, a specific binding pair member may be provided for each analyte. For instance, the specific binding assay may be an antigen-antibody assay. As another example, the specific binding assay may be a nucleic acid hybridization assay. The nucleic acid hybridization assay may be a DNA hybridization assay, or an RNA hybridization assay, in various examples.

At block 1004, the sample may be contacted with specific binding pair members under conditions in which analytes will stably bind with their specific binding pair member but will not bind to other specific binding pair members and in which stably bound specific binding pairs are capable of being detected by emitting a light signal. For instance, the binding pair members may be bound to a solid substrate. The solid substrate may comprise a plurality of beads. For instance, the beads may be labeled, e.g., by a bar code. In some examples, the beads may be magnetic. Additionally, in some examples, two or more specific binding pair members may be bound to different physical locations on the solid substrate.

At block 1006, the method may include providing light (e.g., by a light source such as the light source 804) to the plurality of specific binding pair members and analytes over a period of time wherein stably bound specific binding pairs will emit a light signal.

At block 1008, a plurality of images, each including the plurality of analytes, may be captured at a plurality of durations by one or more photodetectors (e.g., the image detector 806).

For a first analyte of the plurality of analytes, the method 1000 may proceed from block 1008 to blocks 1010-1014, and for a second (different) analyte of the plurality of analytes, the method 1000 may proceed from block 1008 to blocks 1016-1020. In some examples, the method 1000 may proceed in a similar manner with a third analyte, fourth analyte, etc., of the plurality of analytes.

At block 1010, for the first analyte of the plurality of analytes of the sample, the method 1000 may include determining a first range of integration durations of the light signal associated with the presence of the first analyte. For instance, the first range may be calculated based on a signal-concentration response curve for the first analyte. At block 1012, a first image that is captured at an integration duration within the first range of integration durations may be identified, and at block 1014, the first image may be analyzed to determine the presence (or absence) of the first analyte.

At block 1016, for the second analyte of the plurality of analytes of the sample, the method 1000 may include determining a second range of integration durations of the light signal associated with the presence of the second analyte. For instance, the second range may be calculated based on a signal-concentration response curve for the second analyte. At block 1018, a second image that is captured at an integration duration within the second range of integration durations may be identified, and at block 1020, the second image may be analyzed to determine the presence (or absence) of the first analyte.

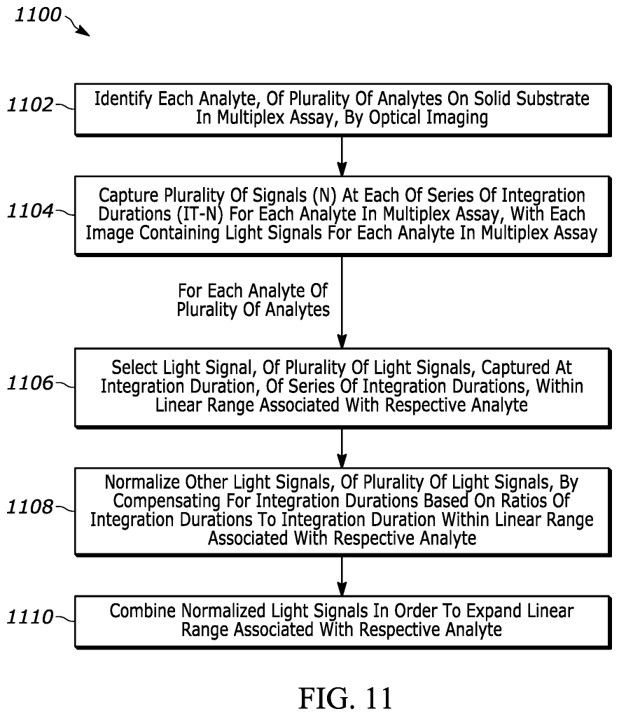

FIG. 11 illustrates a flow diagram of an example method 1100 of detecting a signal associated with the quantitative measurement of an analyte in a multiplex bioassay (e.g., an immunoassay, a molecular assay, etc.), wherein measuring the concentration of an analyte in a sample is indicated by a light signal strength, in accordance with some embodiments provided herein. One or more steps of the method 1100 may be implemented as a set of instructions stored on a computer-readable memory (e.g., memory 814) and executable on one or more processors (e.g., processor 812).

At block 1102, each analyte, of a plurality of analytes (e.g., 2-4096 analytes) on solid substrates in a multiplex bioassay (e.g., sample 805) may be identified by optical imaging (e.g., using the methods discussed above with respect to FIGS. 9 and 10. For example, the solid substrates may include one or more of digitally coded beads, barcoded magnetic beads, color coded microbeads, or microarray settings on a planar surface. For instance, in the example of barcoded solid substrates, each barcoded solid substrate may be immobilized with a specific biomolecular probe which can react with a specific analyte, but not other analytes, in the multiplex assay in the sample. The resulting analyte may be labelled with fluorophore for fluorescence detection.

At block 1104, a plurality of signals (N) (e.g., 2-5 signals) may be captured at each of a series of integration durations (IT-N) (e.g., 0.01 milliseconds to 10 seconds) for each analyte in said multiplex assay. Each image may contain light signals (e.g., fluorescence signals, chemiluminescence signals, etc.) for each analyte in the multiplex assay. These signals may be obtained from optical sensors (e.g., the image detector 806), such as, e.g., CCD, CMOS cameras, photo diodes, photomultiplier tubes, etc.

Blocks 1106-1110 may be performed for each respective analyte of the plurality of analytes. At block 1106, a respective light signal, of a plurality of light signals, that is captured at a respective integration duration, of a series of integration durations, that is within a linear range associated with the respective analyte, may be selected.

At block 1108, the other light signals, of the plurality of light signals, may be normalized by compensating for their integration durations based on a ratio of their integration durations to the respective integration duration that is within the linear range associated with the respective analyte.

At block 1110, the normalized light signals may be combined in order to expand the linear range associated with the respective analyte of the plurality of analytes.

Numerous modifications and variations in the practice of the invention are expected to occur to those skilled in the art upon consideration of the foregoing description on the presently preferred embodiments thereof. Consequently, the only limitations which should be placed upon the scope of the present invention are those that appear in the appended claims.

What is claimed is:

1. A method of conducting an assay for multiple analytes in a sample wherein the presence or absence of each analyte in said sample is indicated by a light signal comprising the steps of:
determining the presence of an analyte by: providing a light source to the sample wherein a light signal is emitted associated with the presence of individual analytes;
capturing, by one or more photodetectors, a plurality of images, each including the plurality of analytes, at a plurality of durations;
for a first analyte of the plurality of analytes,
determining, by one or more processors, a first range of integration durations associated with a light signal associated with the presence of the first analyte;
identifying, by the one or more processors, a first image, of the plurality of images, captured at a duration within the first range of integration durations associated with the light signal associated with the presence of the first analyte; and
analyzing, by the one or more processors, the first image to determine the presence of the first analyte; and
for a second analyte of the plurality of analytes,
determining, by one or more processors, a second range of integration durations associated with the presence of the second analyte;
identifying, by the one or more processors, a second image, of the plurality of images, captured at a duration within the second range of integration durations associated with the light signal associated with the presence of the second analyte; and
analyzing, by the one or more processors, the second image to determine the presence of the second analyte.

2. The method of 1 wherein the first range is calculated based on a signal-concentration response curve for the first analyte”

3. The method of claim 1 wherein the light source is visible light.

4. The method of claim 1 wherein the light source is ultraviolet light.

5. The method of claim 1 wherein the assay is a specific binding assay.

6. A method of conducting a specific binding assay for multiple analytes in a sample wherein the presence or absence of each analyte in said sample is indicated by a light signal comprising:
providing a specific binding pair member for each analyte;
contacting the sample with the specific binding pair members under conditions in which analytes will stably bind with their specific binding pair member but will not bind to other specific binding pair members and in which stably bound specific binding pairs are capable of being detected by emitting a light signal;
determining the presence of stably bound specific binding pair members by:
providing a light source to the plurality of specific binding pair members and analytes over a period of time wherein stably bound specific binding pairs will emit a light signal;
capturing, by one or more photodetectors, a plurality of images, each including the plurality of analytes, at a plurality of durations;
for a first analyte of the plurality of analytes,
determining, by one or more processors, a first range of integration durations associated with a light signal associated with the presence of the first analyte;
identifying, by the one or more processors, a first image, of the plurality of images, captured at a time within the first range of integration durations associated with the light signal associated with the presence of the first analyte; and
analyzing, by the one or more processors, the first image to determine the presence of the first analyte; and
for a second analyte of the plurality of analytes,
determining, by one or more processors, a second range of integration durations associated with the presence of the second analyte;
identifying, by the one or more processors, a second image, of the plurality of images, captured at a time within the second range of integration durations associated with the light signal associated with the presence of the second analyte; and
analyzing, by the one or more processors, the second image to determine the presence of the second analyte.

7. The method of claim 6 wherein said binding pair members are bound to a solid substrate.

8. The method of claim 7 wherein the solid substrate comprises a plurality of beads.

9. The method of claim 6 wherein the beads are labeled.

10. The method of claim 6 wherein the beads are labeled by a bar code.

11. The method of claim 6 wherein the beads are magnetic.

12. The method of claim 6 wherein two or more specific binding pair members are bound to different physical locations on a solid substrate.

13. The method of claim 6 wherein the specific binding assay is an antigen-antibody assay.

14. The method of claim 6 wherein the specific binding assay is a nucleic acid hybridization assay.

15. The method of claim 14 wherein the nucleic acid hybridization assay is a DNA hybridization assay.

16. The method of claim 15 wherein the nucleic acid hybridization assay is an RNA hybridization assay.

17. A method for detecting a signal associated with the quantitative measurement of an analyte in a multiplex bioassay, wherein measuring the concentration of an analyte in a sample is indicated by a light signal strength, comprising:
identifying each analyte, of a plurality of analytes on solid substrates in a multiplex bioassay, by optical imaging;
capturing a plurality of signals, N, at each of a series of integration durations, IT-N, for each analyte in said multiplex assay, wherein each image contains light signals for each analyte in the multiplex assay;
for each respective analyte,
selecting a respective light signal, of the plurality of light signals, captured at a respective integration duration, of the series of integration durations, that is within a linear range associated with the respective analyte;
normalizing the other light signals, of the plurality of light signals by compensating for their integration durations based on a ratio of their integration durations to the respective integration duration that is within the linear range associated with the respective analyte; and
combining the normalized light signals in order to expand the linear range associated with the respective analyte of the plurality of analytes.

18. The method of claim 17, wherein the solid substrates include one or more of digitally coded beads, barcoded magnetic beads, color coded microbeads, or microarray settings on a planar surface.

19. The method for detecting a signal associated with the quantitative measurement of an analyte for multiplex bioassays of claim 18, wherein each barcoded solid substrate is immobilized with a specific biomolecular probe which can react with a specific analyte, but not other analytes, in said multiplex assay in a sample.

20. The method for detecting a signal associated with the quantitative measurement of an analyte for multiplex bioassays of claim 19, wherein the resulting analyte is labelled with fluorophore for fluorescence detection

21. The method for detecting a signal associated with the quantitative measurement of an analyte in a multiplex bioassay of claim 17 wherein said a plurality of signals, N=2-5 signals.

22. The method for detecting a signal associated with the quantitative measurement of an analyte for multiplex bioassays of claim 17 wherein said a series of optical integration durations, IT-N, range from 0.01 millisecond to 10 second.

23. The method for detecting a signal associated with the quantitative measurement of an analyte in a multiplex bioassay comprising for multiplex bioassays of claim 17 wherein said multiple signals are obtained from optical sensors, comprising CCD, CMOS camera, photo diode, and photomultiplier tubes.

24. The method for detecting a signal associated with the quantitative measurement of an analyte in a multiplex bioassay comprising multiplex bioassays of claim 17 wherein the bioassay comprises immunoassays or molecular assays.

25. The method for detecting a signal associated with the quantitative measurement of an analyte in a multiplex bioassay comprising in multiplex bioassays of claim 17 wherein light signals are fluorescence or chemiluminescence signals.

26. The method for detecting a signal associated with the quantitative measurement of an analyte in a multiplex bioassay of claim 19 wherein the number of the plurality of analytes in the multiplex assay range from 2-4096.

27. A system with an analyte-dependent dynamic range for measuring a concentration of each analyte, of a plurality of analytes in a multiplex bioassay, comprising:
a light source configured to provide a series of pulses having different time durations to excite multiple analytes on solid substrates in said sample;
an imaging detector configured to capture a plurality of light image signals (N), having a respective plurality of optical integration durations (ID-N) corresponding to the pulses of said light source, wherein each of said light image contains light signals for each analyte in said multiplex bioassay;
one or more processors; and
one or more memories storing instructions that, when executed by the one or more processors, cause the one or more processors to, for each analyte of the plurality of analytes:
for each respective analyte,
select a respective light signal, of the plurality of light signals, captured at a respective integration duration, of the series of integration durations, that is within a linear range associated with the respective analyte;
normalize the other light signals, of the plurality of light signals by compensating for their integration durations based on a ratio of their integration durations to the respective integration duration that is within the linear range associated with the respective analyte; and
combine the normalized light signals in order to expand the linear range associated with the respective analyte of the plurality of analytes.

28. The system of 27, wherein the solid substrates include one or more of digitally coded beads, barcoded magnetic beads, color coded microbeads, or microarray settings on a planar surface.

29. The system of claim 27, wherein the number of the plurality of analytes in the multiplex assay range from 2-4096.

30. The system of claim 27 wherein for said plurality of signals, N=2-5 signals.

31. The system of claim 27 wherein said series of optical integration durations, ID-N, range from 0.01 millisecond to 10 second.

32. The system of claim 27 wherein said light signals are obtained from optical sensors, or optical camera, which comprises CCD, and CMOS camera.

33. The system of claim 27 wherein the bioassay comprises immunoassays or molecular assays.

34. The system of claim 27 wherein the light signals are fluorescence or chemiluminescence signals.
